(19) United States
(12) Patent Application Publication
(10) Pub. No.: US 2026/0083977 A1
(43) Pub. Date: Mar. 26, 2026

(54) PHOTOTHERAPY DEVICE

(71) Applicant: DONGGUAN LAIGUANG ELECTRONIC TECHNOLOGY CO., LTD., Dongguan City (CN)

(72) Inventor: JIANGXIANG WU, Dongguan City (CN)

(21) Appl. No.: 19/406,732

(22) Filed: Dec. 2, 2025

(30) Foreign Application Priority Data
Oct. 30, 2025 (CN) 202522295195.3

(51) Int. Cl. A61N 5/06; A61N 5/00

(52) U.S. Cl. CPC A61N 5/06; A61N 2005/005; A61N 2005/063; A61N 2005/0633; A61N 2005/0652; A61N 2005/0663

(57) ABSTRACT

The present application provides a phototherapy device, which includes a bracket and a phototherapy mechanism. The bracket is intended to be placed on the ground or a tabletop and is provided with a supporting part. The phototherapy mechanism includes a housing and a lamp module, with the housing detachably connected to the supporting part. A lamp panel is mounted on the housing, and lamp beads are electrically connected to the lamp panel, serving as a light output end of the lamp module. A light-transmitting member is connected to the lamp panel or the housing and covers corresponding lamp beads. The light-transmitting member is exposed at a front of the housing, protecting the lamp beads and allowing the light emitted by the lamp beads to transmit through, to facilitate both the protection of the lamp beads and light transmission by the light-transmitting member.

### CROSS-REFERENCE TO RELATED APPLICATIONS

This application claims priority to Chinese Patent Application No. 202522295195.3, filed on Oct. 30, 2025, the entire disclosures of which are incorporated herein by reference.

### TECHNICAL FIELD

The present application relates to the technical field of phototherapy devices, in particular to a phototherapy device.

### BACKGROUND

The phototherapy device is a physical therapy device that utilizes the action of light. It is applied in various medical and cosmetic fields. In the prior art, existing phototherapy devices include a bracket and a phototherapy mechanism. The bracket is configured to be provided on the ground or a tabletop, and the phototherapy mechanism is connected to the bracket. However, the phototherapy mechanism cannot be disassembled, resulting in poor convenience in assembling and disassembling the phototherapy mechanism of existing phototherapy devices. Meanwhile, the lamp beads of the phototherapy mechanism are exposed to the external environment, leading to poor protection effect on the lamp beads of the phototherapy mechanism in existing phototherapy devices.

### SUMMARY

The purpose of the present application is to provide a phototherapy device, which includes a bracket configured to be provided on the ground or a tabletop. The bracket is provided with a supporting part extending in the vertical direction. The phototherapy mechanism includes at least one phototherapy module, and the phototherapy module includes a housing and a lamp module. The housing is detachably connected to the supporting part. The lamp module includes a lamp panel, lamp beads and a light-transmitting member. The lamp panel is mounted on the housing, the lamp beads are electrically connected to the lamp panel and serve as the light output end of the lamp module. The light-transmitting member is connected to the lamp panel or the housing and covers the corresponding lamp beads. The light-transmitting member is exposed at the front of the housing and protects the lamp beads to allow the light output by the lamp beads to transmit through, so as to facilitate the protection and light transmission of the lamp beads by the light-transmitting member. The housing also has a peripheral protection function, which improves the protection effect of the lamp beads and prolongs the service life of the lamp beads. Meanwhile, the housing can be connected to or detached from the supporting part, avoiding the inability to disassemble the phototherapy mechanism and improving the convenience of assembling and disassembling the phototherapy mechanism. The phototherapy mechanism uses the supporting part to achieve phototherapy at the corresponding height, thereby enhancing the versatility of the phototherapy device.

To achieve the above purpose, the present application provides the following technical solutions: A phototherapy device includes:
a bracket, configured to be provided on a ground or a tabletop, wherein the bracket is provided with a supporting part extending in a vertical direction;
a phototherapy mechanism, comprising at least one phototherapy module;
wherein each phototherapy module includes a housing and a lamp module;
the housing is detachably connected to the supporting part;
the lamp module includes a lamp panel, lamp beads and a light-transmitting member;
the lamp panel is mounted on the housing, the lamp beads are electrically connected to the lamp panel and serve as a light output end of the lamp module;
the light-transmitting member is connected to the lamp panel or the housing and covers corresponding lamp beads; and
the light-transmitting member is exposed at a front of the housing and protects the lamp beads, to allow light output by the lamp beads to transmit through.

### BRIEF DESCRIPTIONS OF THE DRAWINGS

To more clearly illustrate the technical solutions in the embodiments of the present application, the accompanying drawings required for describing the embodiments will be briefly introduced below. Obviously, the accompanying drawings in the following description are only some embodiments of the present application, and those skilled in the art can obtain other accompanying drawings based on these drawings without exerting creative efforts.

For a more comprehensive understanding of the present application and its beneficial effects, the following description will be made in conjunction with the accompanying drawings. In the following description, the same reference numerals denote the same parts.

FIG. 1 is a schematic diagram of a phototherapy device according to an embodiment of the present application.

FIG. 2 is an exploded view of a bracket of the phototherapy device according to an embodiment of the present application.

FIG. 3 is an exploded view of a bracket of the phototherapy device according to a second embodiment of the present application.

FIG. 4 is a front view of the bracket of the phototherapy device according to the second embodiment of the present application.

FIG. 5 is a schematic diagram of a phototherapy mechanism of the phototherapy device according to an embodiment of the present application.

FIG. 6 is another state schematic diagram of the phototherapy mechanism of the phototherapy device according to an embodiment of the present application.

FIG. 7 is a cross-sectional view of a phototherapy module of the phototherapy device according to an embodiment of the present application.

FIG. 8 is an exploded view of the phototherapy module of the phototherapy device according to an embodiment of the present application.

### DETAILED DESCRIPTIONS OF EMBODIMENTS

The technical solutions in the embodiments of the present application will be clearly and completely described below in conjunction with the accompanying drawings in the embodiments of the present application. Obviously, the described embodiments are only a part of the embodiments of the present application, rather than all the embodiments. Based on the embodiments in the present application, all other embodiments obtained by those skilled in the art without creative efforts shall fall within the protection scope of the present application.

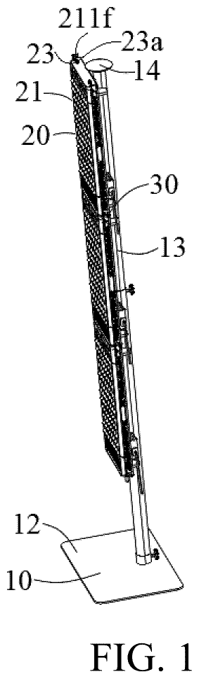

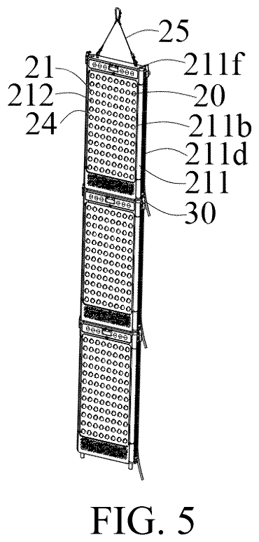

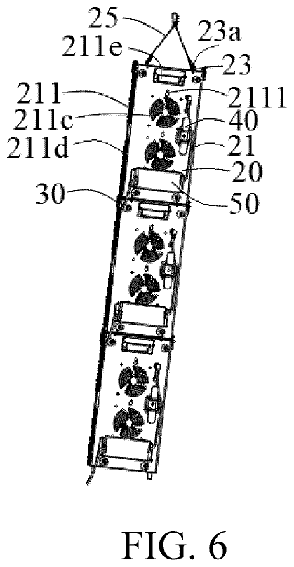

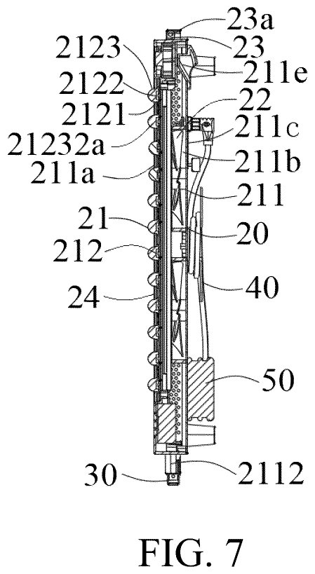

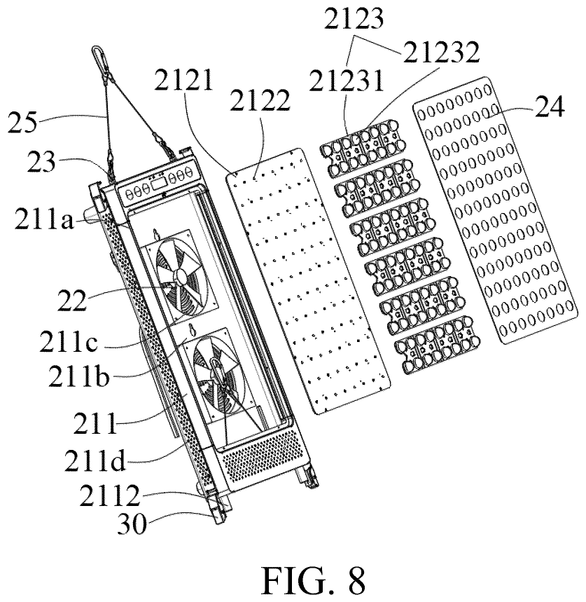

Please refer to FIGS. 1 to 8, an embodiment of the present application provides a phototherapy device 100. The lamp body mounting assembly has the functions and effect such as photobiological activation, anti-inflammation, pain relief, skin treatment, and circadian rhythm adjustment.

Please refer to FIGS. 1 to 8, in the embodiment of the present application, the phototherapy device 100 includes a bracket 10 and a phototherapy mechanism 20. The bracket 10 is configured to be provided on the ground or a tabletop. The bracket 10 is provided with a supporting part 11 extending in the vertical direction. The phototherapy mechanism 20 includes at least one phototherapy module 21, and the phototherapy module 21 includes a housing 211 and a lamp module 212. The housing 211 is detachably connected to the supporting part 11. The lamp module 212 includes a lamp panel 2121, lamp beads 2122 and a light-transmitting member 2123. The lamp panel 2121 is mounted on the housing 211, the lamp beads 2122 are electrically connected to the lamp panel 2121 and serve as the light output end of the lamp module 212. The light-transmitting member 2123 is connected to the lamp panel 2121 or the housing 211 and covers the corresponding lamp beads 2122. The light-transmitting member 2123 is exposed at the front of the housing 211, and protects the lamp beads 2122 to allow the light output by the lamp beads 2122 to transmit through, so as to facilitate the protection and light transmission of the lamp beads 2122 by the light-transmitting member 2123. The housing 211 also has a peripheral protection function, which improves the protection effect of the lamp beads 2122 and prolongs the service life of the lamp beads 2122. Meanwhile, the housing 211 can be connected to or detached from the supporting part 11, avoiding the inability to disassemble the phototherapy mechanism 20 and improving the convenience of assembling and disassembling the phototherapy mechanism 20. The phototherapy mechanism 20 uses the supporting part 11 to achieve phototherapy at the corresponding height, so that the phototherapy mechanism 20 can output light to the user at different heights, thereby enhancing the versatility of the phototherapy device 100.

Please refer to FIGS. 1 to 8, in the embodiment of the present application, the bracket 10 serves as a supporting component of the phototherapy device 100 and is used to support the phototherapy mechanism 20. The bracket 10 is configured to be provided on the ground or a tabletop, so that the phototherapy device 100 can be placed on the ground or a tabletop through the bracket 10. The bracket 10 is provided with a supporting part 11 extending in the vertical direction.

The phototherapy mechanism 20 includes at least one phototherapy module 21, and the phototherapy module 21 includes a housing 211 and a lamp module 212. The housing 211 is detachably connected to the supporting part 11, so that the housing 211 can be connected to or detached from the supporting part 11, thereby enabling the phototherapy mechanism 20 to be assembled and disassembled relative to the bracket 10, avoiding disassemble the phototherapy mechanism 20 cannot be disassembled, thus the convenience of assembling and disassembling the phototherapy mechanism 20 is improved.

The lamp module 212 includes a lamp panel 2121, lamp beads 2122 and a light-transmitting member 2123. The lamp panel 2121 is mounted on the housing 211, so that the lamp module 212 is connected to the housing 211 through the lamp panel 2121. The lamp beads 2122 are electrically connected to the lamp panel 2121 and serve as the light output end of the lamp module 212, so that the lamp beads 2122 output light after being energized. The light-transmitting member 2123 is connected to the lamp panel 2121 or the housing 211 and covers the corresponding lamp beads 2122. The light-transmitting member 2123 is exposed at the front of the housing 211, protects the lamp beads 2122, and allows the light output by the lamp beads 2122 to transmit through, so as to facilitate the protection and light transmission of the lamp beads 2122 by the light-transmitting member 2123. The housing 211 also has a peripheral protection function, which improves the protection effect of the lamp beads 2122 and prolongs the service life of the lamp beads 2122.

Please refer to FIGS. 1 and 5 to 8, in the embodiment of the present application, a plurality of lamp beads 2122 are provided. The plurality of lamp beads 2122 are integrated on the same lamp panel 2121 in an array distribution, so that the light output by the plurality of lamp beads 2122 covers the entire lamp panel 2121, thereby facilitating the plurality of lamp beads 2122 to fully utilize the space of the lamp panel 2121 and ensuring the output light range of the plurality of lamp beads 2122. The lamp beads 2122 are at least one of red light beads, white light beads or RGB light beads.

When the lamp beads 2122 are red light beads, the lamp beads 2122 can output red light. Red light can promote energy production in cells, enhance cell metabolism, increase the generation of adenosine triphosphate (ATP), thereby contributing to tissue repair and regeneration and reducing inflammatory reactions. By promoting blood circulation and lymphatic detoxification, it reduces the release of inflammatory mediators, and can also stimulate nerve endings to promote the release of endogenous analgesic substances, thereby relieving pain. When the lamp beads 2122 are white light beads, the lamp beads 2122 can output white light, which can provide a uniform and bright light environment. When the lamp beads 2122 are RGB light beads, the high brightness and color accuracy of the RGB light beads enable the display device to present more vivid and realistic images, bringing users a more immersive visual experience.

The light-transmitting member 2123 includes a light-transmitting main body 21231 and a plurality of lens parts 21232. The light-transmitting main body 21231 is connected to the lamp panel 2121 and attached to the front side wall of the lamp panel 2121, so that the light-transmitting member 2123 is connected to the lamp panel 2121 through the light-transmitting main body 21231. The plurality of lens parts 21232 are connected to the same light-transmitting main body 21231 and distributed at different positions of the light-transmitting main body 21231 to cover the corresponding lamp beads 2122. The plurality of lens parts 21232 are provided on the light-transmitting main body 21231 in an array distribution, so that the light output by each lamp bead 2122 is converged by each lens part 21232 and then transmitted to the user, avoiding excessive dispersion of the light output by the lamp beads 2122, and realizing the protection of each lamp bead 2122 by each lens part 21232.

Please refer to FIGS. 1 and 5 to 8, in the embodiment of the present application, the light-transmitting main body 21231 is adhered or screwed to the lamp panel 2121. The front side wall of the lamp panel 2121 is provided with an adhesive layer, which covers part of the light-transmitting main body 21231 and fixes the light-transmitting main body 21231 and the front side of the lamp panel 2121, so that the light-transmitting main body 21231 is adhesively connected to the lamp panel 2121 through the adhesive layer, ensuring the position of the light-transmitting main body 21231 relative to the lamp panel 2121 and avoiding accidental deviation of the light-transmitting main body 21231 relative to the lamp panel 2121. The front side wall of the housing 211 is provided with a first opening 211a, which exposes the adhesive layer and the lens part 21232, ensuring that the lens part 21232 is exposed to the external environment, so that the light output by the lamp beads 2122 penetrates to the external environment through the lens part 21232.

Please refer to FIGS. 1 and 5 to 8, in the embodiment of the present application, the adhesive layer covers the front side wall of the lamp panel 2121 and extends to the inner side wall of the housing 211 that accommodates the lamp panel 2121, so as to hermetically connect the lamp panel 2121, ensuring the sealing effect between the housing 211 and the lamp panel 2121 and preventing liquid and dust from entering the inner side of the housing 211 through the connection between the housing 211 and the lamp panel 2121.

Please refer to FIGS. 1 and 5 to 8, in the embodiment of the present application, the lens part 21232 has an arc shape or a round-head shape; the arc-shaped or round-head-shaped lens can effectively focus or diverge light, and the arc-shaped or round-head-shaped lens can make the refraction effect of light at different positions more uniform by reasonably designing the radius of curvature.

The lens part 21232 is provided with a groove 21232a for accommodating the lamp bead 2122, so that the lamp bead 2122 can fully utilize the space of the groove 21232a. A gap is formed between the inner side wall of the groove 21232a and the peripheral side wall of the lamp bead 2122, avoiding contact between the inner side wall of the groove 21232a and the peripheral side wall of the lamp bead 2122, preventing physical damage to the lamp bead 2122 and prolonging the service life of the lamp bead 2122.

Please refer to FIGS. 1 and 5 to 8, in the embodiment of the present application, the housing 211 is provided with an inner cavity 211b, an air inlet hole 211c and at least one heat dissipation hole 211d. The air inlet hole 211c and the heat dissipation hole 211d are distributed on different side walls of the housing 211, realizing heat dissipation in multiple directions and avoiding only unidirectional heat dissipation. The air inlet hole 211c and the heat dissipation hole 211d are communicated with each other through the inner cavity 211b, so that gas flows sequentially along the air inlet hole 211c, the inner cavity 211b and the heat dissipation hole 211d.

Please refer to FIGS. 1 and 5 to 8, the phototherapy mechanism 20 further includes a heat dissipation fan 22. The heat dissipation fan 22 is mounted on the housing 211 and accommodated in the inner cavity 211b, so that the heat dissipation fan 22 is fixed on the inner side of the housing 211 through the inner cavity 211b, making full use of the internal space of the housing 211. The heat dissipation fan 22 is provided relative to the lamp panel 2121 and dissipates heat for the lamp panel 2121. At this time, the heat dissipation fan 22 sucks in external air during operation, and the external air takes away the heat output by the lamp panel 2121 and is discharged to the heat dissipation hole 211d or the external environment of the housing 211 along different directions, so that the heat dissipation effect of the lamp panel 2121 is accelerated through the heat dissipation fan 22, thereby realizing heat dissipation in multiple directions, avoiding only unidirectional heat dissipation and improving the heat dissipation effect of the phototherapy device 100.

Please refer to FIGS. 1 and 5 to 8, in the embodiment of the present application, the air inlet hole 211c is provided on the rear side wall of the housing 211. The heat dissipation holes 211d are provided on the peripheral side wall of the housing 211, a plurality of heat dissipation holes 211d are provided, and the plurality of heat dissipation holes 211d are distributed on different side walls of the housing 211. The air inlet end of the heat dissipation fan 22 faces the air inlet hole 211c. The air outlet end of the heat dissipation fan 22 faces the rear side wall of the lamp panel 2121, and the air output by the air outlet end of the heat dissipation fan 22 takes away the heat output by the lamp panel 2121, so that when the heat dissipation fan 22 is started, the air from the external environment is output toward the rear side wall of the lamp panel 2121 through the air inlet hole 211c, thereby facilitating the contact between the air from the external environment and the rear side wall of the lamp panel 2121. The air takes away the heat output by the lamp panel 2121 and flows to the external environment through the plurality of heat dissipation holes 211d to dissipate heat for the lamp panel 2121.

The plurality of heat dissipation holes 211d are respectively located on opposite side walls of the housing 211, or the plurality of heat dissipation holes 211d are respectively located on adjacent side walls of the housing 211. When the plurality of heat dissipation holes 211d are respectively located on opposite side walls of the housing 211, the plurality of heat dissipation holes 211d are respectively located on the left side wall and the right side wall of the housing 211, so that the air from the external environment takes away the heat output by the lamp panel 2121 and is discharged to the external environment through the left side wall and the right side wall of the housing 211, avoiding only unidirectional heat dissipation and improving the heat dissipation effect of the phototherapy device 100. When the plurality of heat dissipation holes 211d are respectively located on adjacent side walls of the housing 211, the plurality of heat dissipation holes 211d are respectively located on the left side wall and the lower side wall of the housing 211 or the right side wall and the lower side wall of the housing 211, so that the air from the external environment takes away the heat output by the lamp panel 2121 and is discharged to the external environment through the left side wall and the lower side wall of the housing 211 or the right side wall and the lower side wall of the housing 211, avoiding only unidirectional heat dissipation and improving the heat dissipation effect of the phototherapy device 100.

The phototherapy mechanism 20 further includes a resin casting 24. The resin casting 24 sleeves the light-transmitting member 2123 and is hermetically connected to the housing 211, ensuring the sealing effect at the connection between the resin casting 24 and the housing 211, preventing external dust or external liquid from entering the housing 211, improving the shock resistance of the phototherapy mechanism 20 and reducing the noise caused by the heat dissipation fan 22 in the working state.

The phototherapy mechanism 20 further includes a hanging rope 25. The hanging rope 25 is provided at the top of the housing 211 and connected to the hanging hole 23a. The hanging rope 25 is used for hanging on the wall, so that the phototherapy mechanism 20 can be hung on the wall. The phototherapy assembly 100 further includes a wire winder 40 and an adapter 50. The wire winder 40 and the adapter 50 are connected to the rear side of the housing 211. The wire winder 40 is used for winding wires to prevent the wires from being loose.

Please refer to FIGS. 1 and 5 to 8, in the embodiment of the present application, a plurality of phototherapy modules 21 are provided. The plurality of phototherapy modules 21 are all provided on one side of the supporting part 11 and provided in the vertical direction; the height range of the light output by the phototherapy modules 21 is increased by arranging the plurality of phototherapy modules 21. Among two adjacent phototherapy modules 21, the two adjacent housings 211 are connected to each other. The phototherapy device 100 is provided with a swingable second hook 30, which is located between the two adjacent housings 211 and detachably connected to the two adjacent housings 211. This allows the two adjacent housings 211 to be connected to or detached from each other through the second hook 30, thereby facilitating the arrangement of different numbers of phototherapy modules 21 according to different heights of users. Optionally, there are two second hooks 30, which are respectively provided on opposite sides of the housing 211.

One housing 211 is provided with a positioning hole 211f, and the other housing 211 is provided with a positioning post 2112. The positioning post 2112 is positioned and connected to the positioning hole 211f, ensuring the positional accuracy between the two adjacent housings 211. The second hook 30 is provided in the first space enclosed between the two adjacent housings 211, or in the side space of the two adjacent housings 211, so as to fully utilize the space between the two adjacent housings 211.

Please refer to FIGS. 1 to 4, in the embodiment of the present application, the bracket 10 further includes a base part 12 configured to be provided on the ground or a tabletop, which allows the bracket 10 to be placed on the ground or a tabletop through the base part 12. The supporting part 11 is vertically or obliquely provided relative to the base part 12. When the supporting part 11 is vertically provided relative to the base part 12, the phototherapy mechanism 20 can output light to the user along the vertical direction, facilitating the phototherapy mechanism 20 to perform phototherapy on different heights of the user. When the supporting part 11 is obliquely provided relative to the base part 12, the phototherapy mechanism 20 can output light to the user along the oblique direction, facilitating the phototherapy mechanism 20 to perform phototherapy on different angular positions of the user.

The supporting part 11 is liftably connected to the base part 12 and can adjust its height relative to the base part 12. The housing 211 adjusts its height as the supporting part 11 lifts relative to the base part 12, enabling the phototherapy mechanism 20 to adjust its height and output light at different heights. This avoids the inability of the phototherapy mechanism 20 to adjust its height relative to the bracket 10, improving the practicality of the phototherapy device 100.

A plurality of supporting rods 13 are provided between the base part 12 and the supporting part 11, and the plurality of supporting rods 13 are sequentially provided in the vertical direction. The base part 12 is connected to the supporting rod 13 at the lowest end, and the supporting part 11 is connected to the supporting rod 13 at the highest end, so that the height of the supporting part 11 relative to the base part 12 can be adjusted through the plurality of supporting rods 13.

The plurality of supporting rods 13 include a first supporting rod 131 and a second supporting rod 132. The first supporting rod 131 is connected to the base part 12 and is provided with a through hole 131a provided in the vertical direction. The outer contour of the second supporting rod 132 is in clearance fit with the inner contour of the first supporting rod 131, and the second supporting rod 132 is liftably inserted through the through hole 131a. This allows the second supporting rod 132 to lift relative to the first supporting rod 131 through the space of the through hole 131a, so that the supporting part 11 lifts with the lifting of the second supporting rod 132. The second supporting rod 132 is fixedly connected to the first supporting rod 131 to maintain the height of the supporting part 11, ensuring that the adjusted height of the phototherapy mechanism 20 remains unchanged and avoiding accidental movement of the phototherapy mechanism 20.

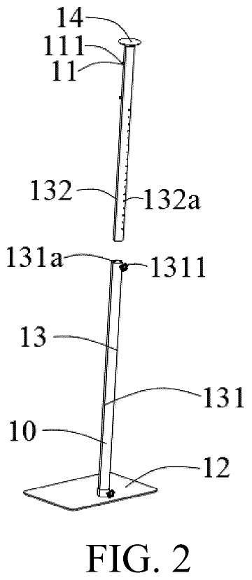

Please refer to FIGS. 1 to 2, in the embodiment of the present application, the second supporting rod 132 is provided with a plurality of through holes 132a provided at intervals along the length direction of the second supporting rod 132. The first supporting rod 131 is connected with a plug-in member 1311, which is movably connected to the first supporting rod 131 to facilitate adjusting the position of the plug-in member 1311 relative to the first supporting rod 131. The plug-in member 1311 can be selectively inserted into each through hole 132a to maintain the heights of the supporting part 11 and the phototherapy mechanism 20 connected to the supporting part 11. This allows the position of the second supporting rod 132 to be restricted by the plug-in member 1311, thereby keeping the height of the phototherapy mechanism 20 on the supporting part 11 unchanged.

Please refer to FIGS. 1 to 2, in the embodiment of the present application, the first supporting rod 131 is provided on the upper side of the base part 12, and the first supporting rod 131 is vertically or obliquely provided relative to the base part 12. When the first supporting rod 131 is vertically provided relative to the base part 12, the phototherapy mechanism 20 can output light to the user along the vertical direction, facilitating the phototherapy mechanism 20 to perform phototherapy on different heights of the user. When the first supporting rod 131 is obliquely provided relative to the base part 12, the phototherapy mechanism 20 can output light to the user along the oblique direction, facilitating the phototherapy mechanism 20 to perform phototherapy on different angular positions of the user. The base part 12 is flat and can be smoothly attached to the ground or a tabletop, and the outer contour of the base part 12 exceeds the projection area of the phototherapy mechanism 20 on the base part 12 along the horizontal direction, this ensures the placement stability of the lamp body mounting assembly 100 and avoids the lamp body mounting assembly 100 from easily toppling over.

The bracket 10 is provided with a circular plate 14, which is provided on the top of the supporting rod 13 at the highest end and connected to the top of the supporting rod 13 at the highest end. The circular plate 14 is used to limit the phototherapy mechanism 20 from exceeding the top of the supporting rod 13 at the highest end, avoiding shaking caused by the high center of gravity of the lamp body mounting assembly 100.

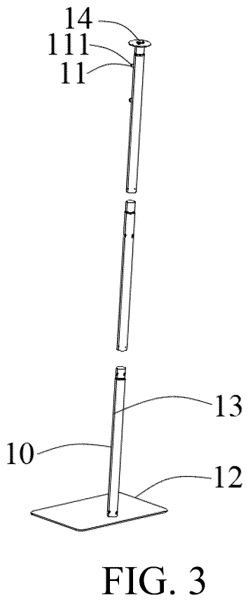

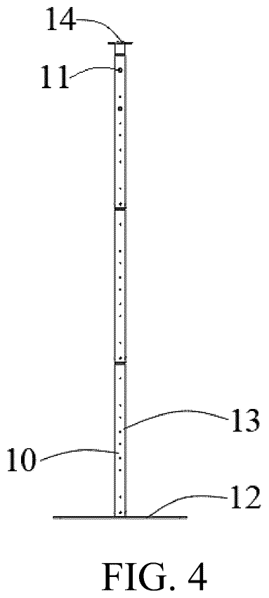

Please refer to FIGS. 3 to 4, in the second embodiment of the present application, the plurality of supporting rods 13 are detachably connected to each other, so that the plurality of supporting rods 13 can be connected to or detached from each other. The height of the supporting part 11 can be adjusted by increasing the number of supporting rods 13. The height of the phototherapy mechanism 20 increases with the increase in the number of supporting rods 13, and decreases with the decrease in the number of supporting rods 13. Specifically, the plurality of supporting rods 13 are detachably connected through plug-in connection.

The housing 211 is provided with a first connecting part 2111. The supporting part 11 is provided with a second connecting part 111, and the second connecting part 111 is connected to the first connecting part 2111 and can be detached from each other. This allows the housing 211 to be connected to or detached from the supporting part 11 through the second connecting part 111 and the first connecting part 2111, thereby realizing the detachable connection of the phototherapy mechanism 20 relative to the bracket 10 and improving the convenience of assembling and disassembling the phototherapy mechanism 20. The connection structure between the second connecting part 111 and the first connecting part 2111 is a quick-release structure, a clamping structure or a hanging structure, so that the second connecting part 111 and the first connecting part 2111 can be detachably connected through the quick-release structure, the clamping structure or the hanging structure.

When the connection structure between the second connecting part 111 and the first connecting part 2111 is a quick-release structure, the second connecting part 111 and the first connecting part 2111 are mutually a quick-release hole and a quick-release post; the quick-release hole is provided with a first hole and a second hole, and the first hole is communicated with the second hole to facilitate the mutual movement of the quick-release post along the first hole and the second hole. The aperture of the first hole is larger than that of the second hole; the quick-release post is inserted through the first hole and located in the second hole to realize the quick-release assembly of the supporting plate and the housing 211. When the quick-release post is located in the first hole, the housing 211 can be detached from the supporting part 11 to realize the detachment of the phototherapy mechanism 20 from the bracket 10. When the quick-release post is located in the second hole, the housing 211 can be connected to the supporting part 11 to realize the fixation of the phototherapy mechanism 20 on the bracket 10.

When the connection structure between the second connecting part 111 and the first connecting part 2111 is a clamping structure, the second connecting part 111 and the first connecting part 2111 are mutually a buckle and a clamping groove, and the buckle is clamped in the clamping groove, so that the second connecting part 111 can be quickly detachably connected to the first connecting part 2111 through the buckle and the clamping groove.

When the connection structure between the second connecting part 111 and the first connecting part 2111 is a hanging structure, the second connecting part 111 and the first connecting part 2111 are mutually a first hook and a hanging hole, and the first hook is inserted into the hanging hole, so that the second connecting part 111 can be quickly detachably connected to the first connecting part 2111 through the first hook and the hanging hole.

The side wall of the housing 211 facing the supporting part 11 is provided with a handle groove 211e, which is arc-shaped and used for the user to grasp. The handle groove 211e is close to the top of the housing 211, so that the user can lift the housing 211 by grasping the handle groove 211e, improving the convenience of carrying the housing 211.

The phototherapy mechanism 20 further includes a hanging arm 23 connected to the upper side wall of the housing 211 and located at the top of the housing 211. The hanging arm 23 is provided with a hanging hole 23a for supporting by an external hanging bracket 10. This allows the phototherapy mechanism 20 to be connected to the external hanging bracket 10 through the hanging arm 23, thereby facilitating the fixation of the phototherapy mechanism 20 on the external hanging bracket 10 without the need for the bracket 10 to support the phototherapy mechanism 20.

The hanging arm 23 and the housing 211 are of an integrated connection structure or a split connection structure. When the hanging arm 23 and the housing 211 are of an integrated connection structure, the connection strength between the hanging arm 23 and the housing 211 is improved, ensuring the connection stability between the hanging arm 23 and the housing 211. When the hanging arm 23 and the housing 211 are of a split connection structure, the hanging arm 23 can be connected to or detached from the housing 211, improving the convenience of assembling and disassembling the hanging arm 23. Optionally, the hanging arm 23 is detachably connected to the housing 211 through a threaded connection.

Compared with the related art, the beneficial effects of the present application are as follows:

The present application provides a phototherapy device 100. The bracket 10 is configured to be provided on the ground or a tabletop. The bracket 10 is provided with a supporting part 11 extending in the vertical direction. The phototherapy mechanism 20 includes at least one phototherapy module 21, and the phototherapy module 21 includes a housing 211 and a lamp module 212. The housing 211 is detachably connected to the supporting part 11. The lamp module 212 includes a lamp panel 2121, lamp beads 2122 and a light-transmitting member 2123. The lamp panel 2121 is mounted on the housing 211, the lamp beads 2122 are electrically connected to the lamp panel 2121 and serve as the light output end of the lamp module 212. The light-transmitting member 2123 is connected to the lamp panel 2121 or the housing 211 and covers the corresponding lamp beads 2122. The light-transmitting member 2123 is exposed at the front of the housing 211, and protects the lamp beads 2122 to allow the light output by the lamp beads 2122 to transmit through, so as to facilitate the protection and light transmission of the lamp beads 2122 by the light-transmitting member 2123. The housing 211 also has a peripheral protection function, which improves the protection effect of the lamp beads 2122 and prolongs the service life of the lamp beads 2122. Meanwhile, the housing 211 can be connected to or detached from the supporting part 11, avoiding the inability to disassemble the phototherapy mechanism 20 and improving the convenience of assembling and disassembling the phototherapy mechanism 20. The phototherapy mechanism 20 uses the supporting part 11 to achieve phototherapy at the corresponding height, so that the phototherapy mechanism 20 can output light to the user at different heights, thereby enhancing the versatility of the phototherapy device 100.

In the above embodiments, the description of each embodiment focuses on different aspects. For parts not described in detail in a certain embodiment, reference may be made to the relevant descriptions of other embodiments.

In the description of the present application, the terms “first” and “second” are only used for descriptive purposes, and cannot be understood as indicating or implying relative importance or implicitly indicating the number of indicated technical features. Thus, the features defined with “first” and “second” may explicitly or implicitly include one or more such features.

Specific examples are used herein to illustrate the principles and implementations of the present application. The descriptions of the above embodiments are only used to help understand the methods and core ideas of the present application; at the same time, for those skilled in the art, according to the ideas of the present application, there will be changes in the specific implementations and application scopes. In summary, the content of this specification should not be construed as limiting the present application.

What is claimed is:

1. A phototherapy device, comprising:
a bracket, configured to be provided on a ground or a tabletop, wherein the bracket is provided with a supporting part extending in a vertical direction;
a phototherapy mechanism, comprising at least one phototherapy module;
wherein each phototherapy module comprises a housing and a lamp module;
the housing is detachably connected to the supporting part;
the lamp module comprises a lamp panel, lamp beads and a light-transmitting member;
the lamp panel is mounted on the housing, the lamp beads are electrically connected to the lamp panel and serve as a light output end of the lamp module;
the light-transmitting member is connected to the lamp panel or the housing and covers corresponding lamp beads; and
the light-transmitting member is exposed at a front of the housing and protects the lamp beads, to allow light output by the lamp beads to transmit through.

2. The phototherapy device according to claim 1, wherein a plurality of lamp beads are integrated on a same lamp panel in an array distribution;
the plurality of lamp beads are at least one of red light beads, white light beads or RGB light beads;
the light-transmitting member comprises a light-transmitting main body and a plurality of lens parts;
the light-transmitting main body is connected to the lamp panel and attached to a front side wall of the lamp panel;
the plurality of lens parts are connected to a same light-transmitting main body and distributed at different positions of the light-transmitting main body to cover a corresponding plurality of lamp beads; and
the plurality of lens parts are provided on the light-transmitting main body in an array distribution.

3. The phototherapy device according to claim 2, wherein the light-transmitting main body is adhered to the lamp panel;
the front side wall of the lamp panel is provided with an adhesive layer, the adhesive layer covers a part of the light-transmitting main body and fixes the light-transmitting main body and a front side of the lamp panel; and
a front side wall of the housing is provided with a first opening exposing the adhesive layer and the plurality of lens parts.

4. The phototherapy device according to claim 3, wherein the adhesive layer covers the front side wall of the lamp panel and extends to an inner side wall of the housing that accommodates the lamp panel, to hermetically connect the lamp panel.

5. The phototherapy device according to claim 2, wherein each lens part has an arc shape or a round-head shape; the lens part is provided with a groove for accommodating the plurality of lamp beads, and a gap is formed between an inner side wall of the groove and a peripheral side wall of the lamp bead.

6. The phototherapy device according to claim 1, wherein the housing is provided with an inner cavity, an air inlet hole and at least one heat dissipation hole;
the air inlet hole and the heat dissipation hole are distributed on different side walls of the housing, and the air inlet hole and the heat dissipation hole are communicated with each other through the inner cavity;
the phototherapy mechanism further comprises a heat dissipation fan, and the heat dissipation fan is mounted on the housing and accommodated in the inner cavity; and
the heat dissipation fan is provided relative to the lamp panel and dissipates heat for the lamp panel, and the heat dissipation fan sucks in external air during operation, the external air takes away heat output by the lamp panel, and is discharged to the heat dissipation hole or an external environment of the housing along different directions.

7. The phototherapy device according to claim 6, wherein the air inlet hole is provided on a rear side wall of the housing;
the heat dissipation holes are provided on a peripheral side wall of the housing, a plurality of heat dissipation holes are distributed on different side walls of the housing; and
an air inlet end of the heat dissipation fan faces the air inlet hole, an air outlet end of the heat dissipation fan faces a rear side wall of the lamp panel, and the air output by the air outlet end of the heat dissipation fan takes away the heat output by the lamp panel.

8. The phototherapy device according to claim 1, wherein a plurality of phototherapy modules are all provided on one side of the supporting part and provided in the vertical direction;
among two adjacent phototherapy modules, two adjacent housings are connected to each other, the phototherapy device is provided with a swingable second hook, and the second hook is located between the two adjacent housings and detachably connected to the two adjacent housings; and
the second hook is provided in a first space enclosed between the two adjacent housings, or the second hook is provided in a side space of the two adjacent housings.

9. The phototherapy device according to claim 1, wherein the bracket further comprises a base part configured to be provided on the ground or the tabletop;
the supporting part is vertically or obliquely provided relative to the base part;
the supporting part is liftably connected to the base part and adjusts its height relative to the base part; and
the housing adjusts the height as the supporting part lifts relative to the base part.

10. The phototherapy device according to claim 9, wherein the base part is provided with a through hole provided in a vertical direction; and
the supporting part is liftably inserted through the through hole and fixedly connected to the base part to maintain a height of the supporting part.

11. The phototherapy device according to claim 7, wherein the plurality of heat dissipation holes are respectively located on opposite side walls of the housing, or the plurality of heat dissipation holes are respectively located on adjacent side walls of the housing.

12. The phototherapy device according to claim 1, wherein the phototherapy mechanism further comprises a resin casting, which sleeves the light-transmitting member and is hermetically connected to the housing.

13. The phototherapy device according to claim 1, wherein the phototherapy mechanism further comprises a hanging arm connected to an upper side wall of the housing and located at a top of the housing;
the hanging arm is provided with a hanging hole for supporting by an external hanging bracket; and
the phototherapy mechanism further comprises a hanging rope provided at the top of the housing and connected to the hanging hole of the hanging arm, and the hanging rope is used for hanging on a wall.

14. The phototherapy device according to claim 9, wherein a plurality of supporting rods are provided between the base part and the supporting part, the plurality of supporting rods are sequentially provided in the vertical direction;
the base part is connected to the supporting rod at a lowest end, and the supporting part is connected to the supporting rod at a highest end;
the plurality of supporting rods comprises a first supporting rod and a second supporting rod, the first supporting rod is connected to the base part, the first supporting rod is provided with a through hole provided in the vertical direction; and
an outer contour of the second supporting rod is in clearance fit with an inner contour of the first supporting rod, and the second supporting rod is liftably inserted through the through hole.

15. The phototherapy device according to claim 14, wherein the second supporting rod is provided with a plurality of through holes provided at intervals along a length direction of the second supporting rod;
the first supporting rod is connected with a plug-in member, which is movably connected to the first supporting rod to facilitate adjusting a position of the plug-in member relative to the first supporting rod; and
the plug-in member is selectively inserted into each through hole to maintain heights of the supporting part and the phototherapy mechanism connected to the supporting part.

16. A phototherapy device, comprising:
a bracket, configured to be provided on a ground or a tabletop; wherein the bracket is provided with a supporting part extending in a vertical direction;
a phototherapy mechanism, comprising at least one phototherapy module;
wherein the phototherapy module comprises a housing and a lamp module;
the housing is detachably connected to the supporting part, and the lamp module comprises a lamp panel, lamp beads and a light-transmitting member;
the lamp panel is mounted on the housing, the lamp beads are electrically connected to the lamp panel and serve as a light output end of the lamp module; and
the light-transmitting member is connected to the lamp panel or the housing and covers corresponding lamp beads.

17. The phototherapy device according to claim 16, wherein the bracket further comprises a base part configured to be provided on the ground or the tabletop;
the supporting part is vertically or obliquely provided relative to the base part;
the supporting part is liftably connected to the base part and adjusts its height relative to the base part; and
the housing adjusts the height as the supporting part lifts relative to the base part.

18. The phototherapy device according to claim 17, wherein a plurality of supporting rods are provided between the base part and the supporting part, the plurality of supporting rods are sequentially provided in the vertical direction;
the base part is connected to the supporting rod at a lowest end, and the supporting part is connected to the supporting rod at a highest end;
the plurality of supporting rods comprise a first supporting rod and a second supporting rod, the first supporting rod is connected to the base part, the first supporting rod is provided with a through hole provided in the vertical direction; and
an outer contour of the second supporting rod is in clearance fit with an inner contour of the first supporting rod, and the second supporting rod is liftably inserted through the through hole.

19. The phototherapy device according to claim 18, wherein the second supporting rod is provided with a plurality of through holes provided at intervals along a length direction of the second supporting rod;
the first supporting rod is connected with a plug-in member, which is movably connected to the first supporting rod to facilitate adjusting a position of the plug-in member relative to the first supporting rod; and
the plug-in member is selectively inserted into each through hole to maintain the heights of the supporting part and the phototherapy mechanism connected to the supporting part.

20. A phototherapy device, comprising:
a bracket, configured to be provided on a ground or a tabletop; wherein the bracket is provided with a supporting part extending in a vertical direction;
a phototherapy mechanism, comprising at least one phototherapy module;
wherein the phototherapy module comprises a housing and a lamp module;
the housing is detachably connected to the supporting part;
the lamp module comprises a lamp panel, lamp beads and a light-transmitting member; the lamp panel is mounted on the housing, the lamp beads are electrically connected to the lamp panel and serve as a light output end of the lamp module;
the light-transmitting member is connected to the lamp panel or the housing and covers corresponding lamp beads;
the phototherapy mechanism further comprises a hanging arm connected to an upper side wall of the housing and located at a top of the housing;
the hanging arm is provided with a hanging hole for supporting by an external hanging bracket; and
the phototherapy mechanism further comprises a hanging rope provided at a top of the housing and connected to a hanging hole of the hanging arm, and the hanging rope is used for hanging on a wall.
